(19) United States
(12) Patent Application Publication
(10) Pub. No.: US 2014/0080924 A1
(43) Pub. Date: Mar. 20, 2014

(54) SELF-EMULSIFYING AND STABILIZING GEL FOR STABILIZING COSMETIC COMPOSITIONS

(71) Applicant: Richard T. RIGG, Cambria Heights, NY (US)

(72) Inventor: Richard T. RIGG, Cambria Heights, NY (US)

(21) Appl. No.: 14/025,911

(22) Filed: Sep. 13, 2013

Related U.S. Application Data
(60) Provisional application No. 61/701,802, filed on Sep. 17, 2012.

(51) Int. Cl. A61K 8/04

(52) U.S. Cl. CPC A61K 8/042
USPC 514/785; 514/788

(57) ABSTRACT

A self-emulsifying and stabilizing gel is disclosed for stabilizing a water-in-oil emulsion or an anhydrous cosmetic to prevent decomposition of said cosmetic compositions, which consists essentially of a shear mixture of:
(i) 2 to 30% by weight of a quaternized clay and a first non-polar organic solvent forming a shear mixture as a first phase; and
(ii) 2 to 50% by weight of a polymeric emulsifier having a hydrophilic/lipophilic balance under 12 in admixture with a second non-polar organic solvent which may be the same or different from the first non-polar organic solvent as a second phase, wherein said polymeric emulsifier serves as a polar activator of the quaternized clay. Preferred polymeric emulsifiers include a polyglyceryl ether or ester or a silicone emulsifier.

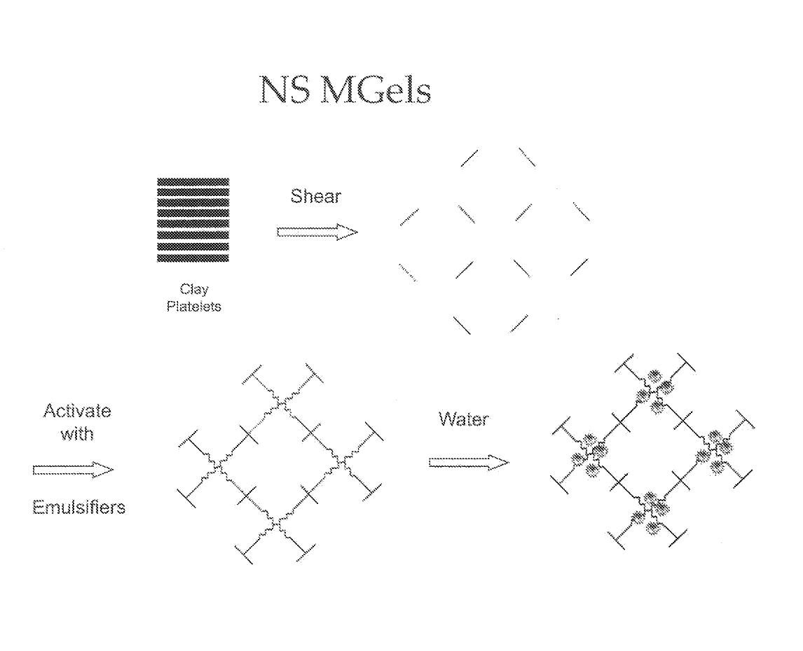

### CROSS REFERENCE TO RELATED APPLICATIONS

This application is related to co-pending U.S. Provisional Application Ser. No. 61/701,802 filed 17 Sep. 2012.

### FIELD OF THE INVENTION

This invention relates to a self-emulsifying and stabilizing gel for stabilizing either an anhydrous cosmetic composition or a cosmetic composition containing a water-in-oil emulsion to prevent decomposition of said cosmetic compositions. The invention further relates to a method for preparing a self-emulsifying and stabilizing gel for stabilizing either an anhydrous cosmetic composition or a cosmetic composition containing a water-in-oil emulsion to prevent decomposition of said cosmetic compositions. This invention further relates to a method for preparing a stabilized cosmetic composition containing a water-in-oil emulsion in order to thicken and provide structure to the oil phase to stabilize the oil phase against separation and oil syneresis by employing the self-emulsifying and stabilizing gel.

### BACKGROUND OF THE INVENTION

Quaternized clays, especially quaternized bentonite or quaternized hectorite, have been used to make gellants/thickeners for use in the cosmetic markets for a very long time. These clays are usually dispersed in an oil to form a very thin dispersion, then activated with high shear in the presence of a polar activator such as propylene carbonate or a short chain alcohol, such as ethanol, to form a viscous gel. The suppliers of quaternized clays specifically direct the user to activate the quaternized clays with one of the abovementioned polar activators before addition of an emulsifier, because, they claim, the emulsifier will interfere with the complete activation of the quaternized clay and lead to an inferior gel. See Southern Clay Products, a Subsidiary of Rockwood Specialties, Inc., Product Bulletin/Tixogel®, Southern Clay Products, Inc., 1212 Church Street, Gonzales, Tex. 78629 and Rheological Additives in Cosmetics, Elements Pure in Cosmetics, Elementis Specialties Consumer Products, PO Box 700, Hightstown, N.J. 08520.

Such pre-made gellants/thickeners are prepared in various anhydrous vehicles and used by manufacturers as thickeners/structurants to prepare various cosmetic products. These premade gels are added to various cosmetic creams, lotions, mascaras, liquid make-up etc. to thicken and stabilize the cosmetic products. The primary functions of the gellants/thickeners include prevention of syneresis of oils, and suspension of pigments to prevent settling. In addition such pre-made gellants/thickeners are used in many other industries, such as the paint and varnish industries.

In the cosmetic field, the pre-made gellants/thickeners are used in two types of products: anhydrous cosmetic compositions and compositions containing a water-in-oil emulsion. In anhydrous systems the gellants/thickeners are used in products such as lipsticks, cosmetic pencils and nail polishes. In lipsticks and pencils, the primary purpose of the gellants/thickeners is to prevent oil syneresis, and in nail polish the gellants/thickeners are used to suspend pigments. In emulsions, the gellants/thickeners are used to thicken/structure the oil phase as well as to keep pigments suspended. And when used in emulsions, the gellants/thickeners are primarily used in water-in-oil (w/o) emulsions to thicken and stabilize the oil phase against separation and oil syneresis. When gelled clays are used in w/o emulsions, they are usually added to the oil phase along with emulsifiers, waxes, etc. to further thicken the emulsions. When emulsifiers are added, they “orient” themselves in a micelle shape in the already-made gel matrix. Once water is added with shear, an emulsion forms.

U.S. Pat. No. 5,843,417 to HANNA et al discloses the use of clay gels activated solely by propylene carbonate or alcohols.

U.S. Pat. No. 8,084,499 to OOTAKE et al specifically points to the problem of stabilizing W/O emulsions, which the inventors believe they have solved by treating a clay mineral with an acylarginine derivative and then activating the pre-gelled clays with propylene carbonate or an alcohol.

U.S. Pat. No. 5,902,591 to HERSTEIN discloses a stable topical emulsion composition containing ascorbic acid in combination with an emulsion composition containing a stabilizing effective amount of an organoclay composition.

US Patent Application 2003/0017123 Alto SCANCARELLA et al discloses water in oil emulsion compositions for making up the eyes and skin comprising at least one film-forming polymer and at least one organic pigment forming the main color component of the composition, said composition being free of inorganic pigments. The water in oil emulsion compositions may further include one or more viscosity modifiers which may include natural or synthetic montmorillonite clays, including bentonite and hectorite, which may be reacted with a quaternary ammonium compound and activated by propylene carbonate.

US Patent Application 2006/0067904 Alto RUSS et al discloses cosmetic compositions that contain water in oil emulsions which may include montmorillonite minerals such as hectorite, bentonite, and quaternized derivatives thereof which are obtained by reacting the minerals with a quaternary ammonium compound, either alone or in combination with a carbonate activator, such as propylene carbonate.

US Patent Application 2010/0331187 A1 to WILLIAMS et al discloses physically stable oil dispersions of bentonite clays and which contain emulsifiers. The emulsifiers, however, are not polymeric emulsifiers, but are alkyl ethoxylates, alkyl ethoxylate phosphate esters, alkyl sulfates, alkyl ammonium salts, castor oil ethoxylates, and mixtures thereof.

None of the prior art water in oil cosmetic compositions containing quaternized clay has adequate stability and these compositions undergo undesired separation of one phase from the other.

### OBJECTS OF THE INVENTION

It is an object of the invention to provide self-emulsifying and stabilizing gel compositions made of quaternized clay that have increased stability and that are less prone to separation than the prior art gellants/thickeners made of quaternized clay.

It is a further object of the invention to provide a method for preparing the self-emulsifying and stabilizing gel compositions made of quaternized clay that have increased stability and that are less prone to separation than the prior art gellants/thickeners made of quaternized clay.

It is a further object of the invention to provide a method for preparing a stabilized cosmetic composition containing a water-in-oil emulsion through the use of the self-emulsifying and stabilizing gel compositions of the present invention.

### SUMMARY OF THE INVENTION

A first feature of the invention includes a self-emulsifying and stabilizing gel for stabilizing a water-in-oil emulsion or an anhydrous cosmetic to prevent decomposition of said cosmetic compositions, which consists essentially of a shear mixture of:

(i) 2 to 30% by weight of a quaternized clay and a first non-polar organic solvent forming a shear mixture as a first phase; and

(ii) 2 to 50% by weight of a polymeric emulsifier having a hydrophilic/lipophilic balance under 12 in admixture with a second non-polar organic solvent which may be the same or different from the first non-polar organic solvent as a second phase, wherein said polymeric emulsifier serves as a polar activator of the quaternized clay.

The self-emulsifying and stabilizing gel may further include

(iii) 2 to 50% by weight of a second polymeric emulsifier having a hydrophilic/lipophilic balance under 12 which may be the same or different from the polymeric emulsifier defined in claim 1 as a third phase.

In the self-emulsifying and stabilizing gel according to the invention, the polymeric emulsifier serves as the primary polar activator of the quaternized clay. In fact the polymeric emulsifier may even serve as the sole polar activator of the quaternized clay although one may add other polar activator compounds as secondary structurants.

Preferably the quaternized clay is quaternized bentonite, quaternized hectorite, quaternized colloidal aluminum silicate, or quaternized montmorillonite.

The quaternized clay is preferably present in an amount of 8 to 25% by weight. The preferred quaternized bentonites include quaternium-90 bentonite, stearalkonium bentonite, and quaternium-18 bentonite. The preferred quaternized hectorites include disteardimonium hectorite and stearalkonium hectorite.

The preferred first and second polymeric emulsifiers having a hydrophilic/lipophilic balance less than 12 include a polyglyceryl ether or ester, especially a polyglyceryl ether or polyglyceryl ester of the Formula (I)

CH₂OR—CHOR—CH₂O—[CH₂CHOR—CH₂O—]ₙCH₂—CHOR—CH₂OR (I)

wherein n is an integer from 1 to 30 and R is H, and in the case where the polymeric emulsifier is a polyglyceryl ether, at least one R must be a C₈ to C₂₂ straight or branched chain saturated, unsaturated or hydroxylated alkyl group and in the case where the polymeric emulsifier is a polyglyceryl ester, at least one R must be —COR′, where R′ is C₈ to C₂₂ straight or branched chain saturated, unsaturated or hydroxylated alkyl group.

In the above mentioned polyglyceryl ethers and polyglyceryl esters the total number of the glyceryl hydroxyalkyl groups that are etherified or esterified ranges between 4 and 400. Examples of the abovementioned polyglyceryl esters include the following: polyglycerol-2 caprate, polyglyceryl-6 argan kernlate, polyglycerol-3 diisostearate, polyglycerol-3 dioleate, polyglycerol-10 heptaoleate, polyglycerol 20 hexacaprylate, polyglyceryl-10 pentaricinoleate, polyglyceryl-10 decahydroxystearate, poyglyceryl-6 polyricinoleate, polyglyceryl-10 dioleate, polyglyceryl-4 dioleate, and polyglyceryl-3 Oleate.

The most preferred polyglyceryl ester emulsifier having a hydrophilic/lipophilic balance less than 12 is polyglyceryl-3 diisostearate.

Another preferred group of polymeric emulsifiers that may serve as either the first polymeric emulsifier, the second polymeric emulsifier or both the first and second polymeric emulsifiers includes silicone emulsifiers, in particular, PEG dimethicones, PPG dimethicones or PEG/PPG dimethicones of the formula (II)

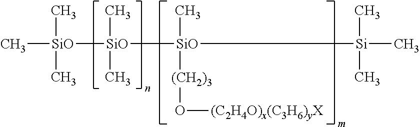

wherein X is selected from the group consisting of hydrogen, alkyl, alkoxy and acyl groups having from about 1 to about 16 carbon atoms, n is from about 1 to about 100, m is from about 1 to about 40, the molecular weight of the residue (C₂H₄O—)ₓ(C₃H₆O—)_{y}X is from about 50 to about 2000, and x and y are each integers ranging from 0 to 25, such that the weight ratio of oxyethylene:oxypropylene is from about 100:0 to about 0:100.

Examples of silicone emulsifiers which are suitable for use in the present compositions include, but are not limited to, dimethicone copolyols, cetyl dimethicone copolyols, laurylmethicone copolyols, including crosslinked forms thereof, and mixtures thereof. Specific examples of preferred silicone emulsifiers include, but are not limited to, dimethicone copolyols such as DC5225C, DC3225C and DC 5000 (commercially available from Dow Corning Corp.); SF1228, SF1328 and SF1528 (commercially available from General Electric Co.); Silwet L-7602 and Silwet L-7622 (commercially available from CKWitco); and KF6015, KF6016 and KF6017 (commercially available from Shin-Etsu); cetyl dimethicone copolyols such as AbilWE-09, Abil WS-08 and Abil EM 90 (commercially available from Evonik Corporation); octyldimethicone ethoxyglucoside copolyols such as Belsil SPG 128 VP (commercially available from Wacker); dimethicone copolyol crosspolymers such as KGS21 (commercially available from Shin-Etsu); and laurylmethicone copolyols such as DC 5200 (commercially available from Dow Corning Corp.).

Preferred silicone emulsifiers include dimethicone copolyol, laurylmethicone copolyol, cetyl dimethicone copolyol, or mixtures thereof.

The present invention uses the emulsifier and optionally the second emulsifier as the primary polar activator for the quaternized clays instead of the recommended polar activators according to the prior art as set forth in the Rockwood brochure, namely, propylene carbonate or short chain alcohols. Though there may be instances where propylene carbonate, short chain alcohols, or other known polar activators may be used as a secondary structurant for the self-emulsifying and stabilizing gel compositions according to the present invention, depending on the solvent, the primary structuring agent remains the polymeric emulsifier or the polymeric emulsifer and the second polymeric emulsifier.

Once the clays are structured using the polymeric emulsifier or emulsifiers, the quaternized clay becomes the self-emulsifying and stabilizing gel.

I have found that water-in-oil emulsions made with these self-emulsifying and stabilizing gels are more stable and less prone to separation than the gels identified in the prior art. The benefits to using emulsions made with the new self-emulsifying and stabilizing gels over the prior art gels include the following:

the new gels according to the invention are self-emulsifying;

the new gels according to the invention make very stable emulsions whether the emulsions are very thick or very thin;

the new gels according to the invention do not require any additional thickener; and

the new gels according to the invention do not require any additional emulsifier(s) other than the polymeric emulsifier(s) in the gel that is(are) used to structure the clay.

I have found that the self-emulsifying and stabilizing gels prepared according to my invention include particles of the clay suspended in the non-polar solvent(s) and the in polymeric emulsifier(s) in the particle size range of 0.2 to 10 μm, preferably 0.5 to 5 μm.

A second feature of the invention includes a method for preparing a self-emulsifying and stabilizing gel for stabilizing a water-in-oil emulsion or an anhydrous cosmetic to prevent decomposition of said cosmetic compositions, which consists essentially of a shear mixture of:

(i) 2 to 30% by weight of a quaternized clay and a first non-polar organic solvent forming a shear mixture as a first phase; and

(ii) 2 to 50% by weight of a polymeric emulsifier having a hydrophilic/lipophilic balance under 12 in admixture with a second non-polar organic solvent which may be the same or different from the first non-polar organic solvent as a second phase, wherein said polymeric emulsifier serves as a polar activator of the quaternized clay, which comprises the steps of:
(a) preparing the first phase by shear mixing of the quaternized bentonite or quaternized hectorite and a first non-polar organic solvent; and
(b) separately preparing the second phase by shear mixing the polymeric emulsifier and the second non-polar organic solvent which may be the same or different from the first non-polar organic solvent, and adding the second phase to the first phase to form a shear mixture of the first and second phases, and optionally adding water.

The method for preparing a self-emulsifying and stabilizing gel may further include:
(c) adding 2 to 50% by weight of a second polymeric emulsifier having a hydrophilic/lipophilic balance under 12 which may be the same or different from the polymeric emulsifier defined in claim 8 to the shear mixture of the first and second phases as a third phase.

A third feature of the invention includes a method for preparing a stabilized cosmetic composition containing a water-in-oil emulsion in order to thicken and provide structure to the oil phase to stabilize the oil phase against separation and oil syneresis, which comprises the steps of:
(a) providing a self-emulsifying and stabilizing gel which consists essentially of a shear mixture of:

(i) 2 to 30% by weight of a quaternized clay and a first non-polar organic solvent forming a shear mixture as a first phase; and

(ii) 2 to 50% by weight of a polymeric emulsifier having a hydrophilic/lipophilic balance under 12 in admixture with a second non-polar organic solvent which may be the same or different from the first non-polar organic solvent as a second phase;
(b) applying a liquid solvent to the self-emulsifying and stabilizing gel to disperse said self-emulsifying and stabilizing gel;
(c) adding a cosmetically effective ingredient and/or a colorant to said dispersed self-emulsifying and stabilizing gel; and
(d) adding water to said dispersed self-emulsifying and stabilizing gel to form the water-in-oil emulsion.

Cosmetically effective ingredients include waxes, emollients, deodorants, anti-perspirants, fragrances, jojoba extract, chamomile extract, rheological additives.

When shear mixing is required to prepare any of the phases or combined phases according to the present invention, either low shear mixing or high shear mixing may be employed in order to properly mix the ingredients. By low shear mixing, I generally mean mixing at a speed of <10 m/s and by high shear mixing I generally mean mixing at a speed of >15 m/s. Usually when low shear mixing is employed, I use a longer mixing time of >30 minutes and when high shear mixing is employed, I use a shorter mixing time of 10 to 15 minutes.

### PREPARATION OF THE SELF-EMULSIFYING GEL ACCORDING TO THE PRESENT INVENTION

### Example 1

Gel Formula and Method of Manufacturing Using a Polymeric Emulsifier as Polar Activator

| Phase | Trade Name | CTFA Name | Formula #1 | Formula #2 |
|---|---|---|---|---|
| A | Tixogel VPV | Quaternium-90 | 20.0 | — |
| | | Bentonite | | |
| A | Permethyl 99A | Isododecane | 57.0 | 57.0 |
| | Bentone 38 | Disteardimonium | — | 20.0 |
| | | Hectorite | | |
| B | Abil EM-90 | Cetyl PEG/ | 10.0 | 10.0 |
| | | PPG-10/1 | | |
| | | Dimethicone | | |
| B | Permethyl 99A | Isododecane | 3.0 | 3.0 |
| C | Lamiform TGI | Polyglyceryl-3 | 10.0 | 10.0 |
| | | Diisostearate | | |

Phase A is combined using a high shear mixer for approx. 40 minutes;
Mix phase B in a separate container and add to phase A slowly with high shear for 10 minutes; and
Add phase C to phrases AB with shear for 5 minutes.

### Preparation of the Gel According to the Prior Art

### Example 2

Gel Formula as Prescribed by Clay Manufacturer

| Phase | Trade Name | CTFA Name | Formula #1 | Formula #1 |
|---|---|---|---|---|
| A | Tixogel VPV | Quaternium-90 | 20.0 | — |
| | | Bentonite | | |
| A | Permethyl 99A | Isododecane | 74.0 | 74.0 |
| A | Bentone 38 | Disteardimonium | — | 20.0 |
| | | Hectorite | | |
| B | Jeffsol Propylene | Propylene | 6.0 | 6.0 |
| | Carbonate | Carbonate | | |

Phase A is combined using a high shear mixer for approx. 40 minutes.
Add phase B to phrases AB with high shear for 5 minutes.
   The typical mixer used to manufacture these gels is a Cowles High Shear Mixer.

### BRIEF DESCRIPTION OF THE DRAWING

The sole FIGURE in this application is a schematic diagram showing the preparation of the self-emulsifying and stabilizing gel according to the invention (NS MGels) starting with the quaternized clay and a non-polar organic solvent, which is subjected to shear mixing, and the resulting mixture is activated with a polymeric emulsifier, and then water is added to form the self-emulsifying and stabilizing gel.

### Preparation of a Soft Texture Long Wearing Low Viscosity Liquid Make-Up According to the Present Invention

### Example 3

### Soft Texture Long Wearing Low Viscosity Liquid Make-Up

### Formula #: NSL11-112-1

| Phase | Ingredient | Supplier | % |
|---|---|---|---|
| A | NS Mgel 1162 | Next Step Lab | 15.0 |
| A | Belsil DM 1 (1 cst Dimethicone) | Wacker | 1.0 |
| A | V sol 5 | Next Step Lab | 32.0 |
| A | DC 556 | Dow Corning | 4.0 |
| B | TIA SSA | Gelest | 8.0 |
| B | YIA SSA | Gelest | 0.80 |
| B | RIA SSA | Gelest | 0.4 |
| B | BIA SSA | Gelest | 0.2 |
| B | Spherilon 10 (Nylon) | Next Step Lab | 2.5 |
| C | Amihope LL | Ajinomoto | 3.0 |
| D | Water | God | 30.0 |
| D | Sodium Chloride | Any | 1.0 |
| D | Neolone 950 | Rohm& Haas | 0.1 |
| D | Butylene Glycol | Any | 2.0 |

### Manufacturing Procedure (Cold Process)

Combine phase A and homomix for 15 minutes until the gel is completely dispersed;
Combine phase B and blend in a blender for 2 minutes;
Add phase B to phase A with homomixing;
Add phase C to phases AB while mixing;
Combine phase D (salt must be fully dissolved). Add phase D to phases ABC while homixing; and
Homomix for 10 minutes after water phase is added and drop the batch.
   Viscosity: RVT, spin #5, 20 rpm, 1 min, RT

| Temp | Initial | 24 hrs | 4 wks | 8 wks | Observation |
|---|---|---|---|---|---|
| RT | 2000 cps | 2000 cps | 2200 cps | 2300 cps | stable |
| 45 C. | — | — | 2100 cps | 2100 cps | stable |
| 50 C. | — | — | 2200 cps | 2200 cps | stable |

I claim:

1. A self-emulsifying and stabilizing gel for stabilizing a water-in-oil emulsion or an anhydrous cosmetic to prevent decomposition of said cosmetic compositions, which consists essentially of a shear mixture of:
(i) 2 to 30% by weight of a quaternized clay and a first non-polar organic solvent forming a shear mixture as a first phase; and
(ii) 2 to 50% by weight of a polymeric emulsifier having a hydrophilic/lipophilic balance under 12 in admixture with a second non-polar organic solvent which may be the same or different from the first non-polar organic solvent as a second phase, wherein said polymeric emulsifier serves as a polar activator of the quaternized clay.

2. The self-emulsifying and stabilizing gel defined in claim 1 which further includes
(iii) 2 to 50% by weight of a second polymeric emulsifier having a hydrophilic/lipophilic balance under 12 which may be the same or different from the polymeric emulsifier defined in claim 1 as a third phase.

3. The self-emulsifying and stabilizing gel defined in claim 1 wherein said polymeric emulsifier serves as the primary polar activator of the quaternized clay.

4. The self-emulsifying and stabilizing gel defined in claim 1 wherein said polymeric emulsifier serves as the sole polar activator of the quaternized clay.

5. The self-emulsifying and stabilizing gel defined in claim 1 wherein the quaternized clay is quaternized bentonite or quaternized hectorite.

6. The self-emulsifying and stabilizing gel defined in claim 5 wherein the quaternized bentonite or quaternized hectorite is present in an amount of 8 to 25% by weight.

7. The self-emulsifying and stabilizing gel defined in claim 5 wherein the quaternized bentonite is quaternium-90 bentonite, stearalkonium bentonite, or quaternium-18 bentonite.

8. The self-emulsifying and stabilizing gel defined in claim 5 wherein the quaternized hectorite is disteardimonium hectorite or stearalkonium hectorite.

9. The self-emulsifying and stabilizing gel defined in claim 1 wherein the polymeric emulsifier is a compound of the Formula (I)
CH₂OR—CHOR—CH₂O—[CH₂CHOR—CH₂O—]ₙCH₂—CHOR—CH₂OR (I)
wherein n is an integer from 1 to 30 and R is H, and in the case where the polymeric emulsifier is a polyglyceryl ether, at least one R must be a C₈ to C₂₂ straight or branched chain saturated, unsaturated or hydroxylated alkyl group and in the case where the polymeric emulsifier is a polyglyceryl ester, at least one R must be —COR′, where R′ is C₈ to C₂₂ straight or branched chain saturated, unsaturated or hydroxylated alkyl group.

10. The self-emulsifying and stabilizing gel defined in claim 1 wherein the polymeric emulsifier is a compound of the Formula (II) 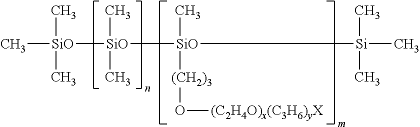
wherein X is selected from the group consisting of hydrogen, alkyl, alkoxy and acyl groups having from about 1 to about 16 carbon atoms, n is from about 1 to about 100, m is from about 1 to about 40, the molecular weight of the residue (C₂H₄O—)ₓ(C₃H₆O—)_{y}X is from about 50 to about 2000, and x and y are each integers ranging from 0 to 25, such that the weight ratio of oxyethylene:oxypropylene is from about 100:0 to about 0:100.

11. The self-emulsifying and stabilizing gel defined in claim 2 wherein the second polymeric emulsifier is polyglyceryl-3 diisostearate.

12. A method for preparing a self-emulsifying and stabilizing gel for stabilizing a water-in-oil emulsion or anhydrous cosmetic to prevent decomposition of said cosmetic compositions, which consists essentially of a shear mixture of:
(i) 2 to 30% by weight of a quaternized clay and a first non-polar organic solvent forming a shear mixture as a first phase; and
(ii) 2 to 50% by weight of a polymeric emulsifier having a hydrophilic/lipophilic balance under 12 in admixture with a second non-polar organic solvent which may be the same or different from the first non-polar organic solvent as a second phase, wherein said polymeric emulsifier serves as a polar activator of the quaternized clay, which comprises the steps of:
(a) preparing the first phase by shear mixing of the quaternized bentonite or quaternized hectorite and a first non-polar organic solvent; and
(b) separately preparing the second phase by shear mixing the polymeric emulsifier and the second non-polar organic solvent which may be the same or different from the first non-polar organic solvent, and adding the second phase to the first phase to form a shear mixture of the first and second phases, and optionally adding water.

13. The method for preparing a self-emulsifying and stabilizing gel defined in claim 11 which further comprises the step of:
(c) adding 2 to 50% by weight of a second polymeric emulsifier having a hydrophilic/lipophilic balance under 12 which may be the same or different from the polymeric emulsifier defined in claim 8 to the shear mixture of the first and second phases as a third phase.

14. A method for preparing a stabilized cosmetic composition containing a water-in-oil emulsion in order to thicken and provide structure to the oil phase to stabilize the oil phase against separation and oil syneresis, which comprises the steps of:
(a) providing a self-emulsifying and stabilizing gel which consists essentially of a shear mixture of:
(i) 2 to 30% by weight of a quaternized clay and a first non-polar organic solvent forming a shear mixture as a first phase; and
(ii) 2 to 50% by weight of a polymeric emulsifier having a hydrophilic/lipophilic balance under 12 in admixture with a second non-polar organic solvent which may be the same or different from the first non-polar organic solvent as a second phase;
(b) applying a liquid solvent to the self-emulsifying and stabilizing gel to disperse said self-emulsifying and stabilizing gel;
(c) adding a cosmetically effective ingredient and/or a colorant to said dispersed self-emulsifying and stabilizing gel; and
(d) adding water to said dispersed self-emulsifying and stabilizing gel to form the water-in-oil emulsion.
